(19) United States
(12) Patent Application Publication
(10) Pub. No.: US 2025/0120904 A1
(43) Pub. Date: Apr. 17, 2025

(54) Method and Apparatus for Drug Delivery to Surgical Margins

(71) Applicant: Oncoblaze LLC, Charleston, SC (US)

(72) Inventor: Dieter Haemmerich, Charleston, SC (US)

(21) Appl. No.: 18/488,954

(22) Filed: Oct. 17, 2023

(51) Int. Cl. A61K 9/00; A61K 9/14; A61K 41/00; A61K 49/00

(52) U.S. Cl. CPC A61K 9/0009; A61K 9/0004; A61K 9/0019; A61K 9/127; A61K 9/14; A61K 41/0033; A61K 41/0042; A61K 41/0052; A61K 49/0017; A61K 49/0093; A61M 5/44; A61M 2205/05; A61M 2205/052; A61M 2205/368

(57) ABSTRACT

A method and apparatus for the targeted delivery of chemotherapy to a surgical cavity, consisting of a triggered nanoparticle encapsulating a therapeutic agent and an energy delivery device that applied trigger energy to the surgical cavity. Following surgical removal of a cancerous tumor, the nanoparticle is administered, and the energy delivery device applies trigger energy to the surgical cavity and proximal tissue. The goal is the delivery of a therapeutic drug dose to cancerous and precancerous cells remaining after surgery, to prevent local tumor recurrence.

### TECHNICAL FIELD

The present invention relates to a method and apparatus for delivery of chemotherapy to normal tissue surrounding a cancerous tumor immediately after surgical removal of that tumor. Specifically, the purpose is treatment of any precancerous cells and cancer cells located in normal tissue remaining after tumor surgery that were not part of the removed tumor, to avoid cancer regrowth.

### BACKGROUND

Cancer, a group of diseases characterized by the uncontrolled growth and spread of abnormal cells, is one of the leading causes of death worldwide. Surgery is a mainstay in the treatment of many cancers. As a primary treatment approach, surgery aims to remove the tumor or cancerous tissue, along with a margin of normal healthy tissue, in order to prevent the spread or recurrence of the disease. The specific surgical approach depends on the type, location, and stage of cancer, as well as the patient's overall health and preferences.

Local tumor recurrence after surgical resection refers to the return of cancer in the same region where the original tumor was removed. Despite advancements in surgical techniques and adjuvant therapies, recurrence remains a significant challenge in cancer treatment. Local recurrence most often results from the presence of individual precancerous cells that will later develop into cancer, or of individual cancer cells in the remaining normal tissue surrounding the original tumor that was removed. To minimize the risk of local recurrence, surgeons aim to remove the tumor with a clear margin of normal healthy tissue (i.e. tissue that does not contain cancerous cells). In some cases surgeons are not able to remove the desired margin of normal tissue due to adjacent sensitive tissues that should not be removed but that likely contain cancer cells. Adjuvant treatments such as radiation, chemotherapy, or immunotherapy are often employed in combination with surgery to target such residual cancer cells and reduce the likelihood of recurrence.

In some cancers such as soft tissue sarcoma and oral cavity cancer, local tumor recurrence rates are particularly high. For soft tissue sarcoma, local recurrence rate depends on width of the surgical margin (i.e., normal tissue removed during surgery), but varies between ^{˜}12 to 38% during 5-year follow-up, and is up to 44% within 10 years [1, 2]. In the most common form of oral cavity cancer—oral squamous cell carcinoma—local recurrence occurs in 20-45% of patients following surgery, and is assumed to result from remaining precancerous cells following surgery [3]. Precancerous cells are genetically altered cells that can become cancer cells at a later point. Local radiation at the area of the surgically removed tumor is commonly used after surgery to reduce the risk of tumor recurrence. While chemotherapy is known to be effective against cancer cells, there is currently no effective method for the targeted delivery of a large dose of chemotherapy to normal tissue surrounding the surgically removed tumor (i.e., the surgical cavity), to reduce the risk of recurrence. While direct application of chemotherapy to the surgical cavity is possible, the chemotherapy then only penetrates 1-2 mm deep into the surrounding tissue [4-6], which is inadequate. For example, Vogelbaum states in a review that “diffusion-driven [drug delivery] techniques . . . only penetrate approximately 1-2 mm from the infusion site before dropping off exponentially” [4]. They further describe as example the implantation of drug-impregnated polymer into the brain tumor resection cavity, describing that “Penetration using this technique is similarly limited to 1-2 mm . . . ”.

Prior art discloses various methods to achieve or enhance delivery of chemotherapy agents to the surgical margin. One prior art method discloses the use of electroporation to enhance cell uptake of chemotherapy injected into the margin tissue, or administered intravenously [7]. Another approach uses high intensity focused ultrasound to either ablate tissue in the margin, or enhance cellular uptake of drugs directly injected into the margin tissue. Yet other approaches use various biomaterials that can be placed in the surgical cavity, that then slowly release therapeutic agents [4, 6, 8]. All these approaches rely on diffusion of chemotherapy agents within tissue. As stated above, such diffusion is very ineffective, and limited to 1-2 mm from the site of drug administration [4-6]. The proposed method and apparatus circumvent this limitation by releasing drug within the vasculature of the margin tissue at depths up to 0.5-3 cm from the surgical cavity surface.

Nanoparticle drug delivery systems (DDS) have been widely research for the targeted delivery of chemotherapy agents to tumors, and include liposomes, micelles, and polymer-based nanoparticles, among others [9-13]. A specific category of DDS are so-called triggered DDS, where release of the encapsulated chemotherapy is initiated by a trigger signal [14]. The trigger that facilitates release depends on the type of triggered DDS, and can include for example temperature, light, ultrasound, electromagnetic fields, or X-rays. Typically, the intended delivery strategy includes extravasation of the nanoparticle DDS, followed by triggered drug release. The vast majority of past DDS for cancer therapy are based on the enhanced permeability and retention (EPR) effect, that enables preferential extravasation of nanoparticle DDS in tumors [10]. The EPR effect is based on the property that tumor vessels are more leaky than normal vessels, combined with a lack of lymphatic drainage present in tumors. This allows for the slow accumulation of nanoparticles in tumors, typically over 24-48 hours [10].

In contrast, the DDS considered in this patent application are based on rapid drug release (within seconds) from DDS within the vessels of the targeted tissue. Recently, such DDS based on so-called intravascular triggered release have been studied more widely, where release occurs primarily within the capillary vessels of the targeted tissue region [15, 16]. Studies by the applicants and others have shown that large quantities of drug can be delivered to a locally targeted tissue regions with triggered nanoparticles based on intravascular triggered release, such as thermosensitive liposomes [15, 17]. The applicants recently established the delivery kinetics of intravascular triggered nanoparticles based on computational models that were validated experimentally [15]. Importantly, they showed that triggered nanoparticles for intravascular triggered release require rapid release within seconds to be effective, since blood only remains for a few seconds within the vessels of the targeted tissue (FIG. 4). Only very few nanoparticles described in the prior art are appropriate for intravascular triggered release [15, 18]. Since the vast majority of prior art is based on slow accumulation of DDS within the tumor, prior art predominantly describes nanoparticles such as thermosensitive liposomes that release the contained drug within minutes to hours [13, 19, 20]. Such release within minutes to hours is inadequate for intravascular triggered delivery considered in this patent.

Various devices have been described in the prior art for exposing cancerous tumors to trigger energy (e.g., hyperthermia) adequate for drug release from triggered nanoparticles. Devices based on radiofrequency fields [21], ultrasound [22, 23], or other energy generating means [24]. The purpose of the proposed device and method is different from these prior art, and is the exposure of the surgical cavity after surgical tumor removal to trigger energy, rather than direct trigger energy application to a deep-seated tumor as described in these prior art. Specifically, the proposed device and method are designed towards exposing a surgical cavity surface and surrounding tissue up to 0.5-3 cm deep.

This patent describes a novel application of triggered nanoparticles, preferably based on intravascular triggered release, to deliver a large chemotherapy dose to the surgical cavity (i.e., the normal tissue surrounding the removed tumor) following surgical tumor resection. The nanoparticles are combined with a trigger energy delivery device specifically designed for exposing the surgical cavity up to 0.5-3 cm deep to trigger energy sufficient to release drug from the nanoparticles. The goal is to kill any precancerous or cancerous cells remaining in the normal tissue surrounding the surgically removed tumor.

### BRIEF DESCRIPTION OF THE DRAWINGS

The provided figures and drawings, which are incorporated into and constitute part of this specifications, illustrate one or more embodiments of the present invention and together with the detailed description, serve to explain the principles and implementation of the present invention.

FIG. 1 shows a flow chart of the intended sequential elements of the procedure.

FIG. 2A shows a tumor that will be surgically removed together with surrounding individual cancerous/precancerous cells and cell islets of cancerous and/or precancerous cells. The dotted line indicates the plane where the surgeon will cut and remove tissue.

FIG. 2B shows the surgical cavity with remaining precancerous and/or cancerous cells, or islets of such cells.

FIG. 2C shows a trigger energy delivery device that exposes the surgical cavity surface up to a certain depth to trigger energy, to release drug from triggered nanoparticles.

FIG. 3 visualizes the intended delivery mechanism of intravascular triggered release, where release of the encapsulated chemotherapy occurs within the vasculature (typically, the capillaries) of the targeted tissue adjacent to the surgical cavity surface. Following chemotherapy release, the drug passes through the vascular wall into tissue and is then taken up by normal, cancerous, and precancerous cells (depending on which cell type is present) with the goal of killing remaining cancerous and/or precancerous cells.

FIG. 4 shows a microscope image of a mouse tissue segment (800×800 um), after injection of a fluorescent contrast agent. These data demonstrate that blood only remains for a few seconds within a particular tissue segment.

FIG. 5A depicts the release kinetics of DDS appropriate for intravascular triggered release, with majority of drug released within 10 s of exposure to trigger energy.

FIG. 5B shows a specific type of thermosensitive liposomes where release was measured by the applicants at body temperature (37° C.), and at hyperthermic temperatures (41 and 43° C.). This type of thermosensitive liposome would be appropriate for the proposed method, where intravascular triggered release is intended.

FIG. 6 shows an embodiment of a hyperthermia device consisting of an infrared laser, an actuated mirror. Tissue temperature is measured either an infrared camera, temperature sensors, or both. If desired, tissue regions where drug delivery is not intended can be protected by adequate material.

FIG. 7 shows the operation of a particular embodiment. After the system obtains an image of the surgical cavity, the user selects a region on this image where drug delivery is intended. In this particular embodiment, heat-sensitive nanoparticles (e.g., thermosensitive liposomes) are employed. Thus, the user also selects a target temperature and heating duration. The system then operates laser and mirrors to expose the tissue surface indicated by the user to uniform temperature.

FIG. 8 shows a graph depicting how convective cooling of the surgical cavity surface can be employed to enhance penetration depth of temperatures required for drug release (here, assumed above 40° C.), and thus increase the tissue depth where drug release occurs.

FIG. 9 depicts fluorescence images from a prior study by the applicants, where drug uptake surrounding a hyperthermia probe is visualized by increasing fluorescence, here shown during 30 min of hyperthermia application.

### DETAILED DESCRIPTION

### A. Overview

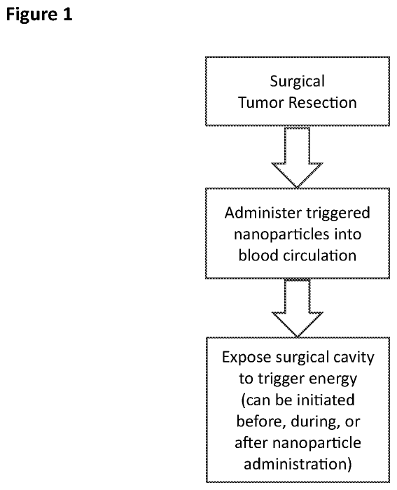

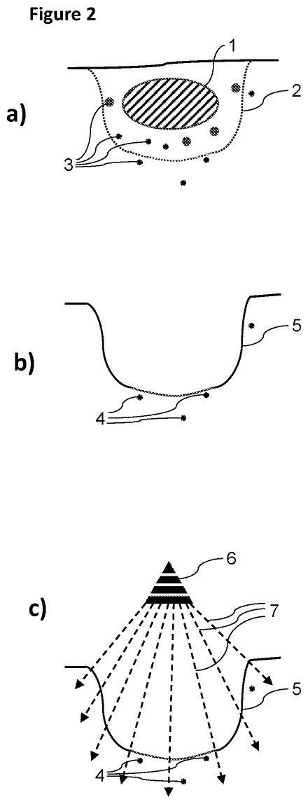

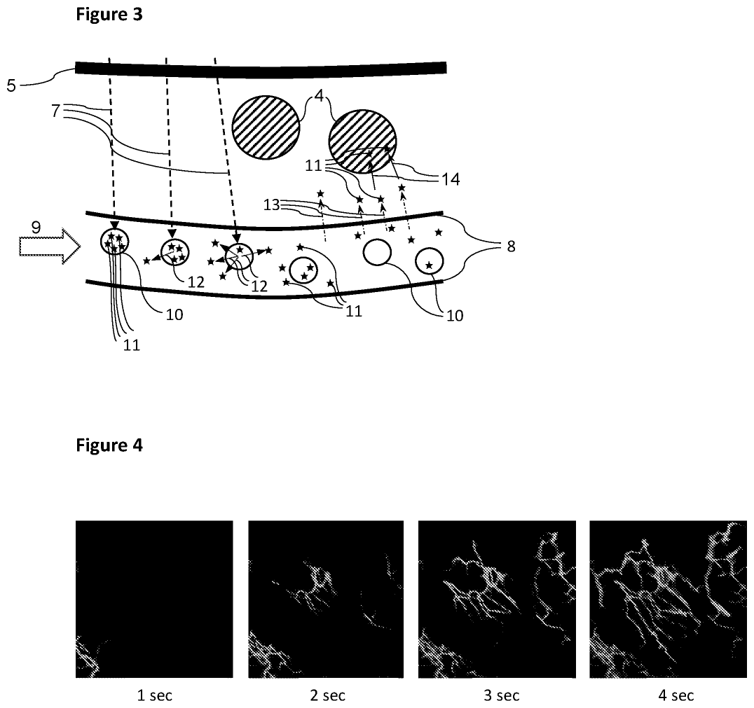

The present invention provides methods and apparatus for the targeted chemotherapy delivery to treat precancerous (cells that may later become cancerous), and cancerous cells remaining in the normal tissue margin, i.e. adjacent to a tumor that has been removed by surgery. Either immediately after surgery, or at a later point, one or more chemotherapy agents encapsulated in triggered nanoparticles are administered to a patient by conventional methods such as intravenous infusion. These nanoparticles will circulate for extended duration in the patient's blood, preferably for at least 30 minutes. Starting either before, during, or following this triggered nanoparticle administration, the surgical cavity is exposed to the trigger signal to induce localized release of the chemotherapy agent (FIG. 1, 2), ideally within 0.5-3 cm of the surgical cavity surface. Preferentially, nanoparticles based on intravascular triggered release will be employed (FIG. 3)—i.e., nanoparticles that release substantial drug amounts while passing through the microvasculature of the target tissue, which means typically within a few seconds (FIG. 4). In a preferred embodiment, heat-triggered nanoparticles such as thermosensitive liposomes will be used; thus, hyperthermia in the range ideal for chemotherapy release from those heat-triggered nanoparticles (typically ^{˜}40-45° C. for thermosensitive liposomes) will be applied to the surgical cavity and deeper surrounding tissue, up to 3 cm deep. Ideally, the trigger energy magnitude (e.g., hyperthermia) will be adequate to trigger drug release from nanoparticles within a 0.5-3 cm wide rim of tissue adjacent to the surgical cavity (FIG. 2c). E.g., if thermosensitive liposomes are used, the goal is to heat a 0.5-3 cm wide rim of tissue to temperatures optimal for release (typically ^{˜}40-45° C.).

### B. Triggered Nanoparticles

Triggered nanoparticles, also known as stimuli-responsive nanoparticles, are a more recent technology in the field of DDS. These nanoparticles can change their properties (e.g., size or porosity) in response to specific stimuli, allowing for controlled and targeted delivery of therapeutic agents. This approach can significantly enhance the efficacy of treatments and minimize side effects, particularly in applications such as cancer therapy. Different types of triggered nanoparticles have been described in prior art, each responding to different triggers [14, 15]. Here, we will focus on nanoparticles that respond to externally applied stimuli. Such external stimuli include light, temperature (thermosensitive), electromagnetic fields, or ultrasound. Triggered nanoparticles allow for high precision in drug delivery, as the release of the therapeutic agent can be controlled spatially (only at the disease site) and temporally (at a specific time) (FIG. 9).

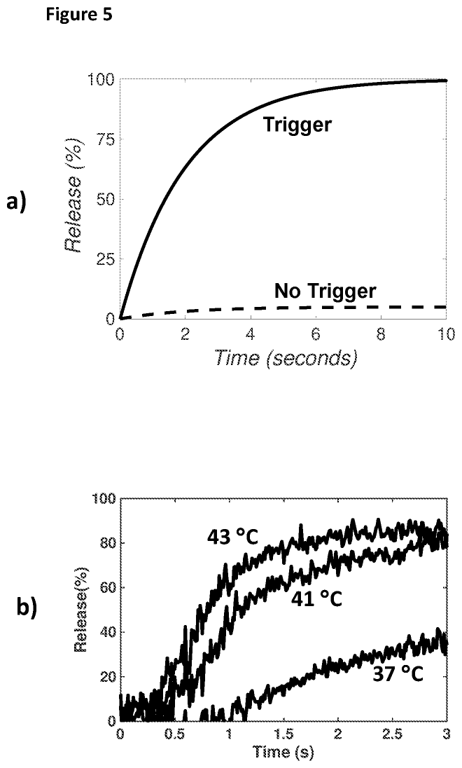

A specific category of triggered nanoparticles that have received more attention in recent years are intra-vascular triggered nanoparticles, as described in detail by the applicants [15]. Intravascular-triggered release from such nanoparticles entails that the release of the encapsulated drug occurs predominantly within the vasculature (typically the capillary vessels) of the targeted tissue region (FIG. 3). In this approach, the systemic circulation serves as reservoir of encapsulated (non-bioavailable) drug, and drug is released (becomes bioavailable) in the vessels of the targeted tissue region where the trigger is applied. Intravascular-triggered nanoparticles require very rapid release of the encapsulated therapeutic agent (e.g., chemotherapy) within a few seconds as demonstrated recently by the applicants [15, 18] (FIG. 5), since blood only remains for a few seconds within a particular tissue segment (FIG. 4). Very few available triggered nanoparticles can achieve such rapid release, and thus most prior art does not describe nanoparticles intended for intravascular triggered release [15, 18]. Such rapid release is required because the nanoparticles circulate through tissue via the blood stream. Blood with nanoparticles remains within any targeted tissue region only for a few seconds (FIG. 4) [15, 18], and has been measured in many human tumors and other tissues. This time is for example ^{˜}2 s for primary liver cancer, ^{˜}3 s for head and neck and prostate tumors, and ^{˜}11 s for kidney cancer [18]. The most prominent intra-vascular triggered nanoparticle in the prior art are thermosensitive liposomes (FIG. 5b). Only a limited number (less than 10) of liposome formulations that are ideal for intravascular-triggered release (i.e., that release most of the contained drug within less than 10 s), are known based on a recent review [18]. Studies by the applicants indicate that even release within minutes is not adequate, as then only a tiny fraction of drug is released while the nanoparticles pass through the vasculature during a few seconds (FIG. 4) [15].

Major advantages of intravascular-triggered nanoparticles include: (1) ability to deliver a large drug dose compared to other nanoparticles [18]; and (2) control of the delivered dose by adjusting the duration of the trigger signal: as long as the trigger is applied, nanoparticle-encapsulated drug enters the target region where drug is then released (FIG. 9). In prior studies, the applicants have shown for thermosensitive liposomes that there is a correlation between hyperthermia duration and drug amount delivered to tumors [17].

Ideally, intra-vascular triggered nanoparticles are combined with drugs that are rapidly taken up by tissue, once released; any released drug not taken up within the tissue region of release will return to systemic circulation and will then be delivered throughout the body. The fraction of drug that is taken up by targeted tissue while passing through tissue vasculature once is often termed ‘extraction fraction’ or ‘extraction ratio’. Chemotherapy drugs with extraction ratios above 0.3 (i.e., >30% of the drug are taken up by targeted tissue) such as doxorubicin, idarubicin, or gemcitabine are preferable, as described by the applicants in recent research [15, 18, 25].

Since thermosensitive liposomes are the most prominent intravascular triggered nanoparticle system [15, 18], a preferred embodiment of the proposed method uses thermosensitive liposomes designed for intravascular triggered release.

Further, in a preferred embodiment such thermosensitive liposomes will encapsulate chemotherapy agents with high extraction ratio (i.e., above 0.3), as described in recent studies and review articles by the applicants [15, 18, 25].

While the majority of triggered particles are ^{˜}100 nm in size and thus termed ‘nanoparticles’, for purposes of the intended embodiments we also consider larger particles (e.g., microparticles that are >1 um in diameter) that are appropriate for intra-vascular triggered release (e.g., ultrasound-triggered microbubbles).

Since the intended delivery strategy is intravascular triggered release, the nanoparticles need to be administered directly or indirectly to the systemic blood circulation, e.g. by intravenous injection or infusion.

### C. Devices for Triggering Release

Depending on the type of the triggered nanoparticle, an appropriate device for applying the release trigger energy to the targeted tissue is required. Examples of devices include focused ultrasound transducers for ultrasound triggered nano- and micro-particles; laser for light-triggered nanoparticles; electromagnetic field generators (e.g., coils) for nanoparticles triggered by electromagnetic fields; and various hyperthermia devices for nanoparticles triggered by heat/temperature. Several such devices have been described in the prior art [13, 23, 24, 26], but none of these prior art devices have been specifically designed for applying trigger energy to a surgical cavity and surrounding tissue.

For application of the trigger signal to a surgical cavity and with optimal tissue penetration (0.5-3 cm), an infrared light source is used as device for applying the release trigger in a preferred embodiment. Advantageously, light can be applied without contact to the surgical cavity and can achieve tissue penetration of 1-2 cm at infrared wavelengths (temperature penetration may be even higher, due to thermal diffusion). Thus, either light-triggered nanoparticles or temperature sensitive nanoparticles would be used in a preferred embodiment using infrared-based devices to trigger drug release.

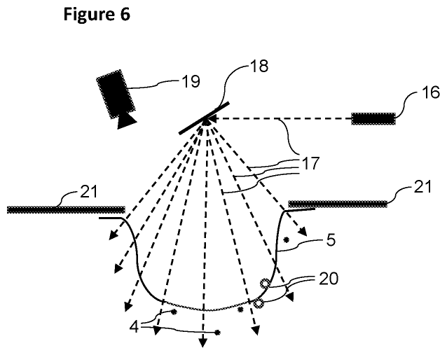

Thus, in a preferred embodiment, either infrared laser or other infrared sources will be employed to target infrared radiation to the surgical cavity and deeper tissues without contact. If infrared lasers are employed, electronically, mechanically, or electro-mechanically actuated mirrors may be used to scan the targeted tissue surface without exposing surrounding tissue outside the cavity (FIG. 6). Alternatively, non-targeted tissue may be covered by adequate material to avoid heating.

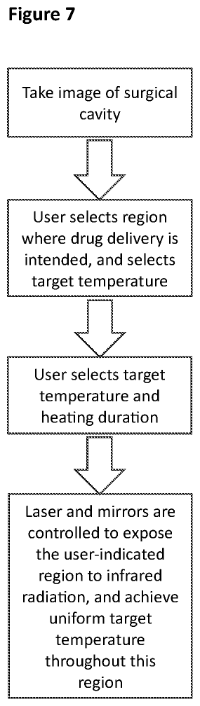

When thermosensitive nanoparticles are employed, monitoring of tissue temperature is important to ensure ideal temperature for drug release is achieved throughout the surgical cavity. Such temperature monitoring can be facilitated either by infrared cameras, or by temperature sensors (e.g., thermocouples, thermistors) placed in contact with the tissue, or below the tissue surface. In a preferred embodiment, temperature feedback from such temperature sensing methods will be employed to modulate power or use pulse width modulation of the infrared device to employ feedback control of tissue heating, and to keep tissue surface temperature within a targeted temperature range. This enables a system where the user selects the target region on an image obtained of the surgical cavity of a patient, and the system automatically exposes the selected target region uniformly to the desired temperature range that facilitates localized drug release (FIG. 7).

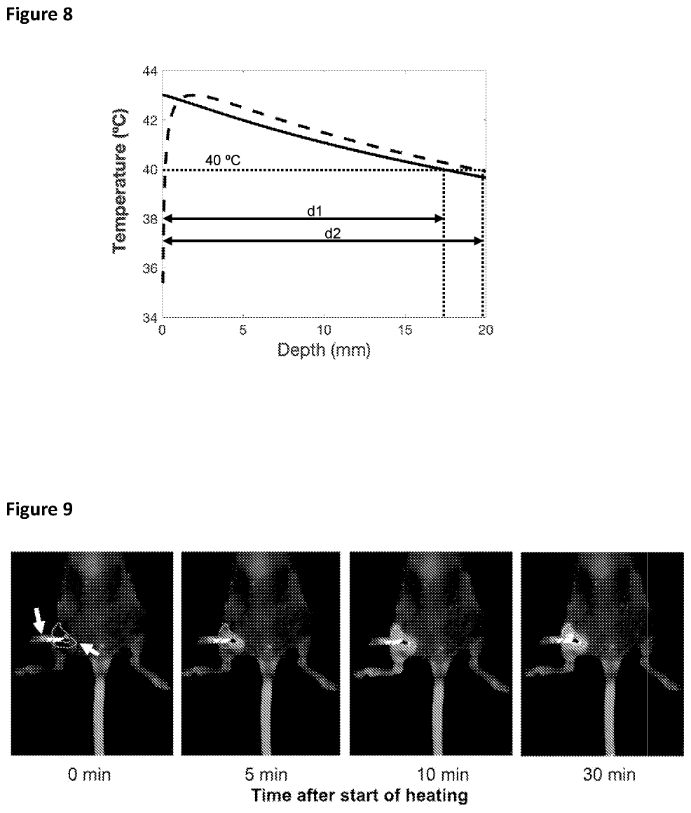

In the proposed heating scenario (FIG. 6), tissue temperature is typically highest at the tissue surface and drops off towards body temperature with increasing tissue depth (FIG. 8). Since release from triggered nanoparticles such as thermosensitive liposomes typically occurs above a certain threshold temperature (e.g., ^{˜}40° C.), drug release is limited to a certain tissue depth (d1 in FIG. 8). If desirable, the depth penetration of temperature, and of drug release can be increased by convective cooling of the tissue surface (d2 in FIG. 8), a method that is known to persons of skill in the art to enhance penetration of tissue heating for interstitial probes [27].

### D. Monitoring of Drug Delivery by Intraoperative Fluorescence Imaging

Intraoperative fluorescence imaging is an emerging technology that enhances the visibility of tissues during surgery, providing surgeons with real-time guidance and improving the accuracy of procedures [28]. Several such intraoperative imaging systems have been described in the prior art [29, 30]. This technique relies on the use of fluorescent probes or dyes, which are administered prior to or during surgery, and a special imaging system that can detect the emitted fluorescence. In the context of cancer surgery, this method can help distinguish cancerous tissues from healthy ones. Certain fluorescent agents are preferentially taken up by cancer cells or are activated only in the presence of cancer, illuminating the tumor when viewed under the imaging system.

In the proposed method and apparatus, intraoperative fluorescence imaging will be employed to monitor location and amount of drug delivered to the surgical cavity, as optional additional component. Such fluorescence imaging requires either naturally fluorescent drugs (such as e.g., doxorubicin, idarubicin, methotrexate), or drugs that have been labeled by fluorophores. Prior art describes the use of fluorescence imaging to visualize fluorescent probes, drugs and nanoparticles following delivery [28-32]. The applicants described in a recent study for the first time the ability to use fluorescence imaging to monitor drug delivery of intravascular triggered thermosensitive liposomes in real-time, during triggered release [17]—i.e., during drug delivery (FIG. 9). In this context, real-time means, that location and amount of drug release could be monitored while the trigger (i.e., hyperthermia) was applied. This enabled visualization of the continuous uptake of drug by the target tissue during delivery (i.e., during heating). This is in contrast to prior studies where release was visualized after delivery was complete [31, 32]. The proposed real-time monitoring enables the user (e.g. physician, medical physicist or nurse) to monitor drug delivery during the procedure, and for example keep applying the trigger until a desired (therapeutically effective) amount of drug has been delivered. This is possible since with intravascular triggered nanoparticles, drug delivery continues to take place as long as the trigger is applied (FIG. 9) [17]. In addition, fluorescence monitoring can ensure that chemotherapy drug is delivered to all desired regions of the surgical cavity. If necessary, trigger energy intensity and/or location can be adjusted to achieve delivery to all desired tissue regions.

### E. Embodiments of Apparatus and Methods for Targeted Chemotherapy Delivery

FIG. 1 illustrates an overview of a method for the targeted delivery of chemotherapy to the surgical cavity. Following surgical removal of a cancerous tumors by conventional methods, triggered drug delivery particles are administered into the systemic blood circulation, e.g., by intravenous infusion. The triggered nanoparticles can include any of those described in the prior art as indicated earlier—and as mentioned, larger microparticles may also be employed instead of the more common nanoparticles. Specific examples include various light-triggered nanoparticles, polymer-based nanoparticles that release drugs triggered by hyperthermia, nanoparticles that can be activated by magnetic or electric fields, and liposomes triggered by X-ray radiation [14, 15]. For purposes of the described embodiments, the nanoparticles are designed for intravascular triggered release. That means that in a preferred embodiment, most of the drug is released within less than 10 seconds from the particles once the particles are exposed to the trigger energy. FIG. 5a shows a plot of the release kinetics of such a nanoparticle designed for intravascular triggered delivery, and FIG. 5b shows release kinetics measured in a particular type of thermosensitive liposomes [33]. The vast majority of the nanoparticles described in the prior art are not suited or designed for intravascular triggered delivery as most nanoparticles release much more slowly, within minutes to hours rather than seconds [18]. This is because these prior art nanoparticles are based on the enhanced permeability and retention (EPR) mechanism, which take place over many hours [10].

Referring now to FIG. 2a, a cancerous tumor 1—such as for example soft tissue sarcoma or oral cavity tumors—is embedded in normal tissue. The proposed embodiments are only applicable for tumors amenable to surgical removal. The tumor 1 is surrounded by individual precancerous or cancer cells and/or small clusters of cancer cells 3. The tissue that is intended to be surgically removed (‘resected’) is indicated by a dotted line 2. The dotted line includes normal tissue surrounding the tumor 1, with the intent of removing all precancerous and cancerous cancer cells, and cluster of precancerous and cancer cells 3.

FIG. 2b shows the tissue remaining immediately after surgical tumor removal. Several precancerous or cancerous cells or clusters of such cells 4 are unintentionally remaining after surgery. These remaining precancerous or cancer cells will likely result in local tumor regrowth, or local tumor recurrence. The surgical cavity 5 indicates the normal tissue surface remaining after surgery. As indicated in FIG. 1, triggered nanoparticles are administered after surgical tumor removal is completed in a preferred embodiment. While the nanoparticles may also be administered before beginning of the surgery or during surgery, this is often disadvantageous since many nanoparticles slowly leak the drug after administration, reducing the drug available for triggered release.

Referring now to FIG. 2c, an energy delivery device 6 is employed to deliver the specific type of energy (‘trigger energy’) 7 required by the triggered nanoparticles for release, to the surgical cavity 5. The trigger energy may either directly induce triggered release, or induce release indirectly. For example, for heat-triggered nanoparticles, the energy delivery device 6 may emit a form of energy that induces tissue heating—for example microwaves, ultrasound, or infrared radiation. Drug release is then induced indirectly due to tissue being heated by this energy. As indicated, in a preferred embodiment, the energy penetrates into the normal tissue such that the remaining precancerous or cancerous cells and precancer/cancer cell clusters 4 are exposed to the trigger energy. Typically, a penetration depth of ^{˜}0.5-3 cm is most desirable since the cancer cells 4 are usually located within less than 0.5-3 cm from the surgical cavity surface 5.

The purpose of the trigger energy 7 is not the direct exposure of the cancer cells 4 to this energy, but the purpose is triggering drug release from the nanoparticles. Referring now to FIG. 3, a microscopic (^{˜}100 um wide) tissue section adjacent to the surgical cavity 5 is shown magnified. Two individual precancer or cancer cells 4 are shown near the surgical cavity surface 5. In addition, a small blood vessel (e.g., capillary) is represented by vessel walls 8. The blood direction in the vessel is indicated by arrow 9. Triggered nanoparticles 10 filled with chemotherapeutic drug 11 are carried by blood through the vessel. The stars 11 indicate individual drug molecules. Once the nanoparticles 10 are exposed to trigger energy 7 of adequate magnitude, the nanoparticles start releasing the contained drug 11 within the vessel. The arrows 12 indicate this triggered release of the drug 11. As the nanoparticles 10 are carried by blood flow along the vessel 8 (from left to right), progressively more and more drug 11 is released. Once drug 11 has been released, the drug crosses the vessel wall as indicated by arrows 13. Finally, the drug 11 is taken up by precancer or cancer cells 4 to induce the intended cell killing. The goal is to release adequate amounts of drug 11 inside the vessels adjacent to precancer/cancer cells 4 near the surgical cavity surface 5, to kill most or all cancer cells 4 and prevent tumor regrowth. Since blood only remains for a few seconds within vessels of a particular tissue segment, nanoparticles require rapid release. FIG. 4 shows an image sequency at second intervals, visualizing the distribution of a contrast agent throughout the capillaries of a small tissue segment; these data demonstrate that blood only remains for a few seconds within the capillaries of a particular tissue segment. The delivery mechanism indicated in FIG. 3 is termed ‘intravascular triggered release’.

Recent experiments and computer simulations by the investigators demonstrate that the time for which the trigger energy 7 is applied, is of primary importance [17, 18]. In the described method, the systemic circulation serves as large reservoir of non-bioavailable drug that becomes bioavailable once the nanoparticles enter the tissue where the release energy is applied to. As long as the release energy is applied, new nanoparticles 10 with drug 11 enter the targeted tissue region and release the drug 11. I.e., the longer the trigger energy 7 is applied, the more drug 11 is released near the surgical cavity 5, and more drug is taken up by cancer cells 4. Thus, in a preferred embodiment, the duration of trigger energy 7 is selected to achieve delivery of drug amounts required for complete cell kill of cancer cells 4.

The type of drug 11 encapsulated in nanoparticles 10 is also of importance. Specifically, recent experiments by the inventors demonstrate that preferred drugs are those that rapidly cross the vessel wall 8, and that are furthermore rapidly taken up by cancer cells 4. A parameter that describes how fast a drug is taken up by tissue is the ‘extraction fraction’ or ‘extraction ratio’ [15, 18]. This extraction fraction describes the fraction of the drug that passes through the vessel wall while carried by blood through the capillary vessel of a particular tissue segment. In a preferred embodiment, the drugs employed have an extraction fraction of 0.3 or higher; i.e., >=30% of drug 11 passes/diffuses across the wall of 8, during a single pass through the vessel. Such drugs with extraction ratio >0.3 include the chemotherapy agents doxorubicin, idarubicin, and gemcitabine, among others [18].

For a triggered nanoparticle to be suitable for intravascular triggered release, substantial amounts of drug need to be released within seconds as discussed earlier (FIG. 5a). One of the best suited triggered nanoparticles for intravascular triggered delivery include some recent formulations of thermosensitive liposomes, since several formulations are available with sufficiently rapid release [18]. Here, hyperthermia serves as trigger energy to induce drug release. For example, the inventors have characterized the release of a formulation of thermosensitive liposomes encapsulating doxorubicin and demonstrated that above ^{˜}41° C., these liposomes release most of the contained drug within ^{˜}2 seconds (FIG. 5b), and are therefore optimal for intravascular triggered release applications. Thus, in another preferred embodiment, thermosensitive liposomes with rapid release such that the majority of the drug is released within less than 10 seconds are employed as triggered nanoparticles.

Referring now again to FIG. 2, when thermosensitive liposomes are employed, the energy device 6 needs to emit a type of energy 7 that induces hyperthermia in the normal tissue adjacent to the surgical cavity 5. Ideal forms of energy include those that can be applied without contact, for example infrared radiation. Ideally, the radiation is applied to the surgical cavity and not to surrounding tissue distant from the cavity 5. FIG. 6 shows a preferred embodiment of a hyperthermia system where an infrared laser 16 is combined with an actuated mirror 18, where the mirror is designed to be controlled by electromechanical means known to persons of skill in the art. A computer-controlled system controls laser activation (e.g., power or on-off timing (i.e., pulse width modulation) and mirror position to expose the whole surgical cavity to infrared radiation. An infrared camera 19 is used to monitor temperature distribution at the tissue surface of the surgical cavity 5, and this temperature information is used as feedback for the computer-controlled system, to achieve uniform temperature throughout the surgical cavity surface. Alternatively or in addition, one or more temperature sensors 20 such as thermistors or thermocouples may be placed at, or adjacent to the cavity surface 5 to monitor temperature and serve for feedback control. During infrared laser application, a temperature gradient will develop such that temperature will drop off with increasing tissue depth from surface of surgical cavity 5, towards body temperature (^{˜}37° C.). Preferably, tissue within up to ^{˜}0.5-3 cm from the cavity surface will be exposed to temperatures adequate for drug release (e.g., above ^{˜}40° C. for typical thermosensitive liposomes). FIG. 8 shows a typical tissue temperature profile when the surgical cavity tissue surface is exposed to heat. Thus, the target temperature measured by infrared camera 19 at the tissue surface of surgical cavity 5 will ideally be selected such that the threshold temperature where substantial release triggered nanoparticles occurs is within ^{˜}0.5-3 cm from the cavity surface 5. The tissue surface of surgical cavity 5 may optionally be cooled by convection with air or other gases to enhance penetration depth of tissue heating (FIG. 8). Sheets 21 made of material opaque to infrared radiation may be employed to shield tissue regions where no exposure and no drug release are desired. Such protection is particularly desirable in other embodiments that provide less accurate spatial control than lasers.

FIG. 7 shows a flow diagram indicating operation of the computer-controlled hyperthermia system shown in FIG. 6. First, an image is obtained of the surgical cavity and surrounding tissue—either by the infrared camera 19 (if the camera has the ability to take conventional photographic images), or optionally by a separate photographic camera. In the latter case, image registration methods known to a person skilled in the art may be employed to register images of the two cameras to each other. Next, the user selects the region where drug delivery is intended—for example, by outlining the surgical cavity on the image with a mouse, touch screen, or other appropriate input methods. In addition, the user may indicate the desired target temperature at the tissue surface, as well as heating duration. Based on these user inputs, the computer-controlled system will control laser and mirror to expose the region selected by the user to the desired target temperature, for the indicated duration. Real-time infrared image data obtained by infrared camera 19 and/or temperature sensors 20 will be used to achieve the desired temperature throughout the user-selected region. Longer heating duration will result in larger amount of drug delivery (FIG. 9) [17].

As additional optional component of the proposed method, a fluorescence imaging system as known to a person skilled in the art will be employed in combination with the system components described above. In a preferred embodiment, fluorescent drugs (e.g., doxorubicin or idarubicin) or fluorescently-tagged drugs will be used to enable fluorescence imaging. Prior studies by the inventors have demonstrated that real-time fluorescence imaging can visualize and monitor drug delivery during heating of tissue, when fluorescent drugs such as doxorubicin are encapsulated in thermosensitive liposomes (FIG. 9) [17]. I.e., the location and amount of drug delivery can be monitored and potentially adjusted in real-time, by adjusting tissue heating via control of the hyperthermia device. The fluorescence imaging system may thus be used to visualize drug delivery in real-time, during heating—for example by overlay of the fluorescence image on a photographic image of the surgical cavity. Alternatively, the fluorescence image data may be used to control the hyperthermia device and adjust heating duration and location such that adequate drug amounts are delivered throughout the surgical cavity.

In particular, it is intended that the present invention not be limited to the embodiments and figures contained herein, but include modifications of those embodiments including portions of the embodiments and combinations of elements of different embodiments as come within the scope of the claims listed below. For example, focused infrared radiation may be provided by other methods than lasers known to persons skilled in the art, such as heat lamps combined with appropriate optical lenses and/or mirrors. A surgical cavity may exhibit various geometries, and may for example be more spherical in case of brain tumors or be very shallow (i.e., not represent a ‘cavity’ in the literal sense) in case of oral cavity cancers.

### CITED REFERENCES

[1] V. Kainhofer, et al., (2016) European Journal of Surgical Oncology (EJSO) 42(6):899-906.
[2] K. R. Gundle, et al., (2018) Journal of clinical oncology: official journal of the American Society of Clinical Oncology 36(7):704-709.
[3] C. R. Anderson, et al., (2015) Oral Oncology 51(5):464-469.
[4] M. A. Vogelbaum, (2005) Journal of Neuro-Oncology 73(1):57-69.
[5] O. Jonas, et al., (2015) Science Translational Medicine 7(284):284ra57-284ra57.
[6] F. Qian, et al., (2003) J Control Release 91(1-2):157-66.
[7] U.S. Pat. App. Nr. 20210308451
[8] C. V. Rahman, et al., (2013) PLOS ONE 8(10):e77435.
[9] S. Sindhwani, et al., (2020) Nature Materials 19(5):566-575.
[10] D. Rosenblum, et al., (2018) Nat Commun 9(1):1410.
[11] U.S. WO2002000298A1
[12] U.S. Pat. No. 5,720,976
[13] U.S. Pat. App. Nr. US20100068260A1
[14] Y. Wang, et al., (2017) Nature Reviews Materials 2(6):17020.
[15] T. L. M. ten Hagen, et al., (2021) Communications Biology 4(1):920.
[16] A. A. Manzoor, et al., (2012) Cancer Research 72(21):5566-75.
[17] A. Motamarry, et al., (2019) International Journal of Hyperthermia 36(1):817-826.
[18] D. Haemmerich, et al., Review of the Delivery Kinetics of Thermosensitive Liposomes, Cancers, 2023.
[19] U.S. Pat. No. 7,769,423
[20] U.S. Pat. No. 6,726,925
[21] U.S. Pat. App. Nr. US20170209579A1
[22] U.S. Pat. App. Nr. US6623430B1
[23] U.S. Pat. App. Nr. US20140135681A1
[24] U.S. Pat. App. Nr. US7056318B2
[25] K. K. Ramajayam, et al., (2022) Int J Hyperthermia 39(1):998-1009.
[26] U.S. Pat. App. Nr. US20190262057A1
[27] U.S. Pat. No. 8,882,755
[28] J. S. D. Mieog, et al., (2022) Nature Reviews Clinical Oncology 19(1):9-22.
[29] U.S. Pat. No. 9,080,977
[30] U.S. Pat. No. 10,231,626
[31] X. Han, et al., (2019) Nanoscale 11(3):799-819.
[32] O. S. Wolfbeis, (2015) Chemical Society reviews 44(14):4743-68.
[33] A. L. Bredlau, et al., (2018) Drug Deliv 25(1):973-984

1-35. (canceled)

36. A method for treating residual cancerous or precancerous cells following tumor surgery in a patient, the method comprising:
administering nanoparticles loaded with at least one anti-cancer agent to the patient, the nanoparticles being configured for delivery to a surgical cavity; and
applying a release-triggering energy to specified regions within or adjacent to the surgical cavity,
wherein the release-triggering energy is applied before, during, or after nanoparticle administration.

37. The method of claim 36, wherein the nanoparticles comprise thermally triggered nanoparticles configured for triggered release by hyperthermia.

38. The method of claim 37, wherein tissue at a depth in a range from 0.5 cm to 3 cm of a surface of the surgical cavity is exposed to the hyperthermia.

39. The method of claim 38, wherein the hyperthermia is in a range from 40° C. to 50° C.

40. The method of claim 36, wherein the thermally triggered nanoparticles are thermosensitive liposomes.

41. The method of claim 36, wherein the nanoparticles are configured to release more than 50% of the at least one anti-cancer agent within 10 seconds in response to the release-triggering energy.

42. The method of claim 36, wherein the at least one anti-cancer agent has an extraction ratio of more than 0.3 (30%).

43. The method of claim 36, wherein the release-triggering energy has one or both of a predetermined duration or a predetermined magnitude.

44. The method of claim 36, wherein the at least one anti-cancer agent is fluorescent or tagged with a fluorescent marker, the method further comprising fluorescence imaging to visualize a location and a quantity of the anti-cancer agent one or both of during or after the application of the near-infrared energy.

45. The method of claim 36, further comprising:
selecting a drug delivery region including at least a part of the surgical cavity based on an image acquired of the surgical cavity and surrounding tissue; and
exposing the drug delivery region to a predetermined hyperthermic target temperature in a range from 40 to 50° C.

46. A method comprising:
introducing thermosensitive liposomes encapsulating doxorubicin into a bloodstream, wherein the thermosensitive liposomes are configured for triggered intravascular release at temperatures above 40° C.;
targeting near-infrared radiation onto a surgical cavity surface to cause at least some of the near-infrared radiation to penetrate below the surgical cavity surface, wherein the near-infrared radiation is configured to release doxorubicin from the thermosensitive liposomes inside tissue capillaries, and wherein the released doxorubicin is configured for killing residual cancer cells remnant in tissue in a vicinity of the surgical cavity surface;
monitoring tissue temperature and adjusting near-infrared radiation intensity based on the monitored tissue temperature; and
monitoring doxorubicin delivery during near-infrared radiation exposure of the surgical cavity surface by fluorescence imaging.

47. The method of claim 46, wherein tissue at a depth in a range from 0.5 to 2 cm of the surgical cavity surface is heated by the near-infrared radiation to temperatures in a range from 40 to 45° C., for a duration in a range from 10 to 60 minutes.

48. The method of claim 46, wherein the residual cancer cells are associated with soft tissue sarcoma or oral cavity squamous cell carcinoma.

49. The method of claim 46, further comprising selecting a tissue region comprising at least a part of the surgical cavity surface before targeting the near-infrared radiation,
wherein targeting the near-infrared radiation comprises exposing the selected tissue region to the near-infrared radiation to heat the selected tissue and induce release of doxorubicin from the thermosensitive liposome.

50. The method of claim 46, further comprising applying convective air cooling with air surgical cavity surface for exposure of internal tissue to cause hyperthermia adequate for inducing release of doxorubicin from the thermosensitive liposomes.

51. The method of claim 46, wherein the near-infrared radiation is targeted before, during, or after introducing the thermosensitive liposomes into the bloodstream.

52. A method comprising:
administering thermally triggered nanoparticles configured for intravascular triggered release, loaded with at least one anti-cancer agent, into a bloodstream; and
applying near-infrared energy to a tissue surrounding a surgically extracted tumor, wherein the near-infrared energy is configured to release the anti-cancer agent from the nanoparticles in the tissue surrounding the surgically extracted tumor.

53. The method of claim 52, wherein the thermally triggered nanoparticles are thermosensitive liposomes.

54. The method of claim 52, wherein the thermally triggered nanoparticles are configured for intravascular release of more than 50% of the at least one anti-cancer agent within 10 seconds when exposed to temperatures in a range from 40 to 45° C.

55. The method of claim 52, wherein the at least one anti-cancer agent has an extraction ratio of more than 0.3 (30%).

56. The method of claim 52, wherein the at least one anti-cancer agent comprises at least one of doxorubicin or idarubicin.

57. The method of claim 52, wherein the near-infrared energy has one or both of a predetermined duration or a predetermined intensity.

58. The method of claim 52, wherein applying the near-infrared energy is configured to expose tissue at a depth in a range from 0.5 to 3 cm of the surgical cavity surface to hyperthermia in a range from 40 to 50° C.

59. The method of claim 52, further comprising monitoring temperature of the tissue by one or more of an infrared camera or a temperature probe placed at or near the surgical cavity surface.

60. The method of claim 52, further comprising selecting a tissue region comprising at least a part of the surgical cavity surface before targeting the near-infrared radiation based on an image acquired of the tissue before applying the near-infrared radiation,
wherein targeting the near-infrared radiation comprises exposing the selected tissue region to the near-infrared radiation to heat the selected tissue and induce release of the at least one anti-cancer agent from the thermally triggered nanoparticles.

61. The method of claim 52, wherein the at least one anti-cancer agent is fluorescent or tagged with a fluorescent marker, the method further comprising fluorescence imaging to visualize a location and a quantity of the anti-cancer agent one or both of during or after the application of the near-infrared energy.

62. The method of claim 61, further comprising modifying one or more of an intensity, a location, or a duration of the near-infrared radiation based on the fluorescence imaging to promote delivery of the anti-cancer agent to the tissue.

63. A method comprising:
infusing thermally triggered nanoparticles incorporating at least one anti-cancer drug, wherein the nanoparticles are configured for intravascular triggered release, into systemic blood circulation; and
releasing drug from thermally triggered nanoparticles in a tissue surrounding a surgically extracted tumor by applying a drug-releasing energy, wherein the released drug is configured to kill remnant cancer cells.

64. The method of clause 63, further comprising applying the drug-releasing energy by a near-infrared laser.

65. The method of claim 64, wherein the thermally triggered nanoparticles are thermosensitive liposomes configured for intravascular triggered release of more than 50% of the contained chemotherapy agent within less than 5 seconds when exposed to temperatures above 40° C.
